(19) United States
(12) Patent Application Publication
(10) Pub. No.: US 2024/0260876 A1
(43) Pub. Date: Aug. 8, 2024

(54) A MEDICAL STATUS ANALYSIS SYSTEM AND METHOD

(71) Applicant: Eaton Intelligent Power Limited, Dublin 4 (IE)

(72) Inventors: Padhraig RYAN, Kilkenny, Keatingstown (IE); Daniel ZUCCHETTO, Dublin (IE); Om TANDON, Dublin, Dublin 1 (IE)

(21) Appl. No.: 18/566,409

(22) PCT Filed: Jun. 2, 2021
(86) PCT No.: PCT/EP2021/064862

§ 371 Date: Dec. 1, 2023

(51) Int. Cl. A61B 5/22; A61B 5/00

(52) U.S. Cl. CPC A61B 5/225; A61B 5/6895; A61B 5/7267; A61B 2503/10; A61B 2562/0219; A61B 2562/0247

(57) ABSTRACT

A medical status analysis system, comprising a positionable sleeve a distributed array of pressure sensors arranged to detect a pressure applied to the sleeve; an inertial sensor arranged to detect an inertial measurement of an object; and a processor operable to detect an event of interest, by: receiving inertial input data; and determining that the event of interest has occurred and operable to detect a grip of a user on the sleeve; and analyse the grip of the user on the sleeve, by: receiving input data; determining a grip attribute based on the input data; and determining a medical status of the user based on the grip attribute. Finally, the processor is operable to output the medical status corresponding to the user. The medical status analysis system therefore preferably provides a user of the system with meaningful and interpretable data regarding their medical status.

### FIELD OF THE INVENTION

The present invention relates to a medical status analysis system and method and finds particular, although not exclusive, utility in a system and method for providing an athlete, such as a golfer or skier, with feedback related to their medical status based on their grip during use of their sports equipment, such as a golf club or ski.

### BACKGROUND TO THE INVENTION

A change in grip strength during sports that require the use of an instrument, such as a golf club or ski, can be an important indicator of an athlete's medical status. The change in grip strength may arise due to a change in the strength of the user's wrist, palm and/or fingers whilst using the instrument. Hand and wrist morbidity is especially common in older individuals and can result in a degradation in the quality of a user's grip. In particular, hand and wrist osteoarthritis is the leading cause of disability in people aged over 50 years, and negatively impacts grip strength and range of motion. Declining grip strength is a feature of multiple chronic illnesses and is also associated with an increased risk of Alzheimer's disease in older people. In rheumatoid arthritis, a common symptom is reduced finger strength, and reduced range of motion—highlighting the importance of grip exercises, such as golf, to strengthen hands and fingers.

Grip strength may also provide indirect information related to walking difficulties. In particular, a condition leading to a leg and/or spine weakness in a person may lead to the person applying more pressure with their grip in order to grip walking aids, e.g. canes. A small but gradual change in such pressure over time can therefore indicate a worsening of the condition for the person.

Hand, wrist or arm injuries may also impact grip strength. Said injuries may occur during physical activity or unrelated activities.

The measurement of grip strength can provide physicians with a useful insight into a patient's medical wellbeing. However, in typical healthcare settings, assessment of grip strength is underused. It is challenging for physicians and nurses to routinely test grip strength, due to a lack of time during brief consultations.

Therefore, it is desirable to provide a medical status monitoring system and method capable of indicating a medical status of a user based on the user's grip on an object/instrument. Objects and aspects of the present invention seek to provide such a system and method.

### SUMMARY OF THE INVENTION

According to a first aspect of the present invention, there is provided a medical status analysis system comprising: a sleeve positionable, in use, on an object configured to be gripped by a user; a distributed array of pressure sensors arranged to detect a pressure applied to the sleeve; an inertial sensor arranged to detect an inertial measurement of the object; and a processor operable to: detect, with the inertial sensor, an event of interest, by: receiving inertial input data from the inertial sensor; and determining that the event of interest has occurred based on a comparison between the inertial input data and a predetermined inertial threshold; detect, with the array of pressure sensors, a grip of a user on the sleeve; analyse the grip of the user on the sleeve, by: receiving input data from the array of pressure sensors; determining a grip attribute based on the input data; and determining a medical status of the user based on the grip attribute; and output the medical status corresponding to the user.

In the context of the present invention, the term “event of interest” will be understood by the skilled addressee as referring to an event during a session in which it is useful to measure a grip of a user. For example, in a session of golf the event of interest may be when a golf shot occurs. The grip of the user during the golf shot in which a user swings the gold club, a certain threshold of grip strength may be required to enable impact with a golf ball without losing grip of the golf club. Accordingly, input data captured during the event of interest may be more interpretable and/or meaningful than when the user is carrying the golf club for other purposes.

Alternatively, the event of interest may be a propelling action during skiing, in which a threshold grip strength is required to apply a force to the ground via the ski without losing grip of the ski.

Alternatively, an algorithm may be used to identify when an event of interest takes place. Accordingly, the event of interest may be determined without a user manually tagging the input data.

A key advantage of the present invention is that the system may provide a user with feedback related to their medical status based on their grip on an object without having to routinely test grip strength in a healthcare setting. Further advantageously, feedback may be provided during the event of interest which may provide a more interpretable and meaningful set of input data.

The object may be a golf club. The sleeve may be a golf club grip. Accordingly, at least one grip attribute may relate to a user's golf grip. For example, at least one grip attribute may indicate that the user's golf grip has degraded over time. Alternatively, or additionally, the at least one grip attribute may localise a weakness to a particular area of a hand used to grip the golf club.

Alternatively, the object may be another piece of sports equipment, such as a ski, a baseball bat, a tennis racket, a badminton racket, a cricket bat, a hockey stick, a hurley, a lacrosse stick, a table tennis paddle, a fishing rod, or any other known sports equipment configured to be held by a user. Accordingly, a user may obtain some feedback related to their grip of the sports equipment.

Alternatively, the object may be a piece of non-sporting equipment, such as a steering wheel, a trolley handle, a mobile phone, a kitchen knife, a screwdriver or any other known piece of non-sporting equipment configured to be held by a user. Accordingly, a user may obtain some feedback related to their grip of the non-sporting equipment.

The inertial sensor may be any one selected from the range of: an accelerometer; a gyroscope; a tilt tensor; and a magnetometer. The accelerometer may be a microelectronic mechanical accelerometer, a piezoelectric accelerometer, or another form of accelerometer. Alternatively, a combination of the inertial sensors may be selected. Accordingly, the inertial sensor may measure the inertial input data as an acceleration, an angular velocity, and/or a change in strength and direction of a magnetic field in the vicinity of the object. In this way, the inertial input data may be used to determine when an event of interest has occurred. For example, during a golf shot, an increased acceleration of the golf club during the swing may lead to an acceleration of the golf club, which may be measured by the accelerometer. Alternatively, a change in angular velocity of the golf club may be measured by the gyroscope. Alternatively, a change in strength and direction of a magnetic field may be measured by the magnetometer (e.g. orientation of the sensor relative to the earth's magnetic field).

Preferably, the event of interest comprises: inertial input data that has met the predetermined inertial threshold; and a clock data; wherein the clock data corresponds to the inertial input data.

The “clock data” will be understood by the skilled addressee as referring to a measurement of time associated with the inertial input data. Each data point comprised in the inertial input data may comprise an associated time signature based on the clock data. The time signature may be comprised in metadata associated with the inertial input data. In this way, a time frame in which the event of interest has taken place may be determined.

The “predetermined inertial threshold” will be understood by the skilled addressee as being a predetermined threshold to be applied to the inertial input data. The predetermined inertial threshold may be a predetermined acceleration threshold. That is, the predetermined acceleration threshold may be a minimum acceleration value. For example, the minimum acceleration value may be 20 ms². The minimum acceleration value may be less than 20 ms². The minimum acceleration value may be more than 20 ms². Different activities may require a different minimum acceleration value. For example, skiing may require that a ski undergoes a lower acceleration than a golf club during a golf shot. The predetermined inertial threshold may be a predetermined angular velocity threshold. That is, the predetermined angular velocity threshold may be a minimum angular velocity. For example, the minimum angular velocity value may be 6 rad/s. The minimum angular velocity value may be less than 6 rad/s. The minimum angular velocity value may be more than 6 rad/s. Different activities may require a different minimum angular velocity. For example, skiing may require that a ski undergoes a lower angular velocity than a golf club during a golf shot.

The predetermined inertial threshold may comprise a maximum inertial threshold. In this way, inertial input data that is greater than the maximum inertial threshold is not included in the event of interest.

A time series may be generated, by the processor, associated with the inertial input data which has met the predetermined inertial threshold. Accordingly, the event of interest may comprise the time series indicative of when the event of interest occurred.

The input data may comprise a temporal component. The temporal component may indicate a time measurement associated with the input data. Each data point comprised in the input data may comprise an associated time signature. The time signature may be comprised in metadata associated with the input data.

A time range may be generated, by the processor, associated with the input data.

In some embodiments, the processor is further operable to: compare the temporal component of the input data to the clock data of the event of interest; and output event data having a temporal component matching the event clock data. Accordingly, input data having a temporal component that matches the clock data of the event of interest may be output. That is, input data measured by the pressure sensors during the event of interest may be output. Advantageously, the input data measured during the event of interest may be more interpretable and meaningful than input data measured outside the event of interest.

The input data from the array of pressure sensors may be stored on a remote server. The inertial input data from the inertial sensor may also be stored on the remote server.

Input data having a temporal component outside the clock data of the event of interest may be discarded. Advantageously, storage space occupied by input data may be reduced on the remote server. Alternatively, input data having a temporal component outside the clock data of the event of interest may be used to determine a pressure of a user's grip prior to use of an instrument. In this way, a user's normal grip may be determined. Advantageously, the user's normal grip may be used to improve the accuracy of the system. The “user's normal grip” will be understood by the skilled addressee as meaning the grip of the user when using the object outside of the event of interest, for example when the user is carrying the golf club.

Preferably, the grip attribute is determined using the event data having the matching temporal component. In this way, the grip attribute is determined based on input data that may be more interpretable, meaningful, and reproducible. Alternatively, the grip attribute may be determined using the full set of input data.

The grip attribute may be one or more selected from the range of: an average grip strength; a maximum grip strength; a temporal change in grip strength; a longitudinal change in grip strength across a length of the sleeve; and a lateral change in grip strength across a width of the sleeve substantially perpendicular to a length of the sleeve. Said range of grip attributes, used in combination or alone, may provide an indication of a user's grip strength and/or medical status.

The grip attribute may be determined using the input data of an activity. Alternatively or additionally, the grip attribute may be determined using input data received over a plurality of activities. That is, the input data may comprise pressure applied to the sleeve during activities that have occurred during different sessions. The plurality of activities may be the same type of activity, for example golf. Alternatively or additionally, the plurality of activities may be different types of activity, for example golf and skiing.

Preferably, the processor is operable to determine the average grip strength by: averaging the input data over the temporal component. Accordingly, an average grip strength may be determined throughout an activity. Advantageously, the average grip strength may provide an insight into the degree of pressure that the user is capable of applying over a sustained period, which may be influenced by muscle strength, stamina and a duration of recovery periods.

Preferably, the processor is operable to determine the maximum grip strength by: calculating a maximum pressure comprised in the input data over the temporal component. The maximum pressure comprised in the input data may be compared with the average grip strength in order to determine a variation in grip strength. Additionally, the maximum grip strength may provide insight into an instantaneous ability of the user to apply grip pressure, without necessarily considering the effect of stamina or durability of grip pressure.

Preferably, the processor is operable to determine the temporal change in grip strength by: determining a first pressure having a first time stamp corresponding to the temporal component; determining a second pressure having a second time stamp corresponding to the temporal component; and calculating a difference between the first pressure and the second pressure. The first time stamp may correspond to the second time stamp. That is, the first time stamp preferably corresponds to a segment of a first event of interest and the second time stamp preferably corresponds to a segment of a second event of interest, wherein the segments are substantially similar. For example, the first time stamp may correspond to the start of a swing during a first golf shot and the second time stamp may correspond to the start of a swing during a second golf shot. The different events of interest may occur during the same session. Alternatively or additionally, the different events of interest may occur during different sessions. For example, a first event of interest may occur during a golf shot occurring during a first session and a second event of interest may occur during a golf shot occurring during a second session. Accordingly, the difference between the first pressure and the second pressure may measure a change in grip strength between the same segments of two different golf shots. Advantageously, a degradation in grip strength during a session, or between sessions, may be quantified. Alternatively, the effects of rehabilitation or other therapeutic interventions may be characterised by an examination of the temporal change.

Preferably, the processor is operable to determine the longitudinal change in grip strength by: determining a first longitudinal pressure at a first longitudinal position along a longitudinal axis of the sleeve; and determining a second longitudinal pressure at a second longitudinal position spaced from the first longitudinal positon along the said longitudinal axis of the sleeve; wherein determining the longitudinal change in grip strength comprises calculating a difference between the first longitudinal pressure and the second longitudinal pressure. The skilled addressee will understand that the longitudinal axis spans the length of the sleeve. Preferably, a longitudinal distance between the first and second longitudinal positions is greater than a width of the user's fingers. In this way, pressure may be measured for different fingers. The first and second longitudinal positons may share a common axis parallel to or in line with the longitudinal axis. Advantageously, a weakness in grip may be localised to a particular one of the user's fingers and/or palms. For example, the first longitudinal positon may be located adjacent to a midsection of a user's index finger and the second longitudinal positon may be located adjacent to a midsection of the user's ring finger. Preferably, the first and second longitudinal pressures comprise the same temporal component. In this way, the first and second longitudinal pressures are measured at the same time. Advantageously, one (or multiple) longitudinal pressure point(s) may serve as a point of reference for another longitudinal pressure point. For example, golfers may deliberately adopt a less forceful grip for certain shots, and the comparison of multiple longitudinal points may help to distinguish a grip pressure that is deliberately weaker in aggregate, as opposed to an isolated weakness in one particular part of the hand or fingers.

Preferably, the processor is operable to determine the lateral change in grip strength by: determining a first lateral pressure at a first lateral position along an orthogonal axis; and determining a second lateral pressure at a second lateral position; wherein determining the lateral change in grip strength comprises calculating a difference between the first lateral pressure and the second lateral pressure. The skilled addressee will understand that the orthogonal axis is orthogonal to the length of the sleeve. The first and second lateral pressures share a common axis parallel to or in line with the orthogonal axis. Advantageously, a weakness in grip may be localised to a particular portion of one of the user's fingers and/or palms. For example, the first lateral positon may be located adjacent to a midsection of a user's index finger and the second lateral positon may be located adjacent to a tip of the user's index finger. Preferably, the first and second lateral pressures comprise the same temporal component. In this way, the first and second lateral pressures are measured at the same time. Advantageously, one (or multiple) lateral pressure point(s) may serve as a point of reference for another lateral pressure point. For example, golfers may deliberately adopt a less forceful grip for certain shots, and the comparison of multiple lateral points may help to distinguish a grip pressure that is deliberately weaker in aggregate, as opposed to an isolated weakness in one particular part of the hand or fingers.

The remote server may be in communication with at least one other medical status analysis system. In this way, a user may utilise multiple systems and store all input data on the remote server. Advantageously, the detection of a medical issue may be more precise.

Preferably, the processor is adjacent to the sleeve and is operatively connected to: the array of pressure sensors; and the inertial sensor.

The system may be configured to provide data and/or feedback to a user in real time. Alternatively, or additionally, the system may be configured to provide historical data to a user. In this way, the user may track their medical status or recall previous medical statuses saved in the historical data.

The system may further comprise a feedback device. The feedback device may be configured to receive the at least one grip attribute output by the processor. The feedback device may be operable to provide a user gripping the object with feedback related to the grip attribute corresponding to the grip of the user on the sleeve. The feedback device may be operably connected to the processor. The feedback device may be physically or wirelessly connected to the processor. The feedback device may be adjacent the sleeve. For example, the feedback device may be on, embedded into, or under the sleeve. Alternatively, or additionally, the feedback device may be separate from the sleeve and distanced from the object. For example, the feedback device may be positionable on the user away from their hands.

The feedback device may comprise a visual feedback device operable to provide a user gripping the object with visual feedback. The visual feedback may indicate a medical status of the user based on their grip attribute. Alternatively or additionally, the visual feedback may indicate a user's grip attribute. The visual feedback device may comprise a display. The display may be wearable, such as eyewear, or standalone. The visual feedback device may comprise a smart phone or a smart watch. For example, the smart phone or smart watch screen may be used to provide visual feedback.

According to a second aspect of the present invention, there is provided a medical status analysis method comprising the steps: detecting, by an array of pressure sensors, a grip of a user on a sleeve; detecting, by an inertial sensor, an inertial measurement of the object; detecting, with the inertial sensor, an event of interest, by: receiving inertial input data from the inertial sensor; and determining that the event of interest has occurred based on a comparison between the inertial input data and a predetermined inertial threshold; analysing the grip of the user on the sleeve by: receiving input data from the array of pressure sensors; determining a grip attribute based on the input data; determining a medical status of the user based on the grip attribute; and outputting the medical status corresponding to the user.

In some embodiments the medical status analysis system is a grip analysis system.

### BRIEF DESCRIPTION OF THE DRAWINGS

FIG. 1 is a schematic view of a grip analysis system; and

FIG. 2 is a flow diagram showing a method of providing a user with a medical status using the grip analysis system of FIG. 1.

### DETAILED DESCRIPTION

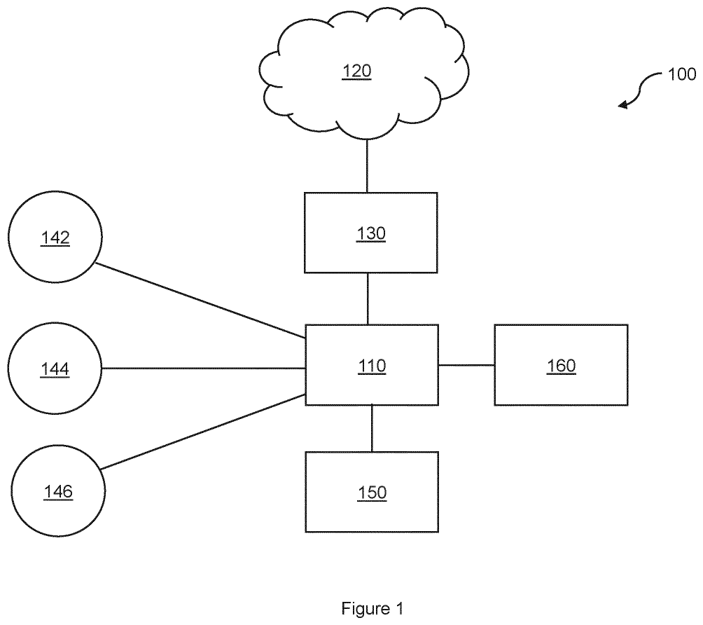

FIG. 1 is a schematic view of a grip analysis system 100. The system includes a processor 110 that is in communication with a cloud-based server 120 via a smart device 130. The processor 110 may be physically or wirelessly connected to the smart device 130 such as a smart phone or a smart watch. For example, the processor 110 and smart device 130 may communicate wirelessly via WiFi or Bluetooth.

The grip analysis system 100 also includes an array of pressure sensors, shown schematically by sensor elements 142, 144, 146. Although only three sensor elements 142, 144, 146 are shown, any number of sensor elements may be provided. For example, 368 sensor elements may be provided in a grid pattern. The array of pressure sensors 140 is configured to be arranged on an object to be gripped by a user, such as a golf club. In this case, the array of pressure sensors 140 may be on, under or embedded in the grip of the golf club or any other connected location. Each sensor element 142, 144, 146 is operable to provide pressure data to the processor 110. Each sensor element 142, 144 146 may also be operable to provide an array position indicative of a position of each sensor element 142, 144, 146 on the sensor array 140.

Furthermore, the grip analysis system 100 also includes a visual feedback device 150. Other types of feedback device 150 are envisaged such as an audible feedback device.

In addition, the grip analysis system 100 includes an inertial sensor 160. The inertial sensor 160 may be an accelerometer operable to provide acceleration data to the processor 110. The inertial sensor 160 may be a gyroscope operable to provide angular velocity data to the processor 110. Other types of feedback device 160 are envisaged such as a magnetometer.

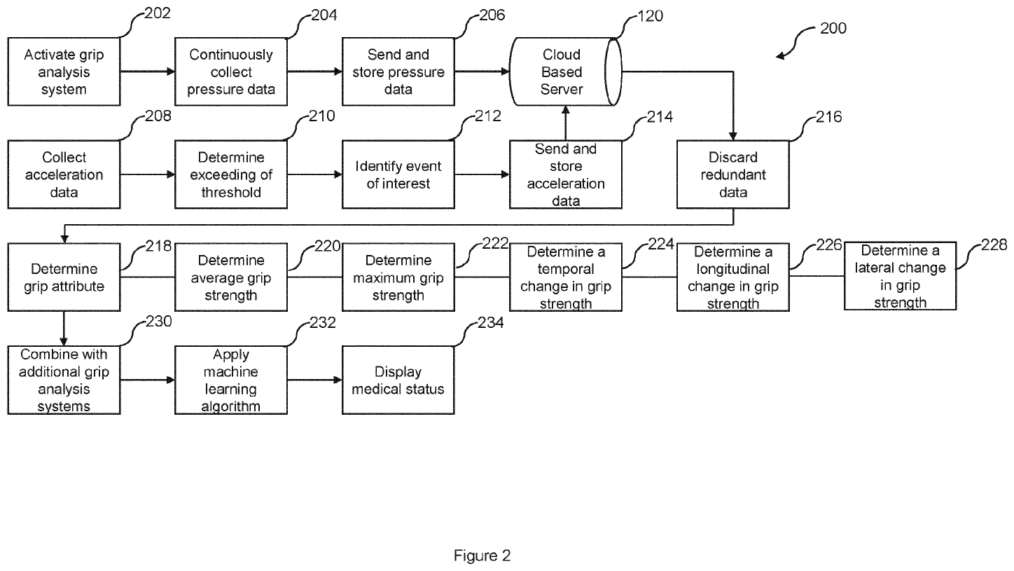

The processor 110 is operable to receive pressure data from the array of pressure sensors 140, receive inertial data from the inertial sensor 160 and process the pressure and inertial data with a method, to be discussed in more detail with reference to FIG. 2, to obtain a medical status. The visual feedback device may be operable to display the medical status.

FIG. 2 is a flow diagram 200 showing an in use method of providing a user with a medical status using the grip analysis system 100 of FIG. 1. In this embodiment, the object to be gripped by the user is a golf club, the inertial sensor 160 is an accelerometer 160 and the inertial data is acceleration data.

The first step, 202 of the method 200 is to activate the grip analysis system 100. The grip analyses system 100 may be activated automatically in response to a user taking a hold of the golf club in a grip and thereby applying a pressure to the sensor array 140. Alternatively, the sensor array 140 may be activated by a switch (not shown) or other activating device. The switch may be operated by the user in order to signify the start of an activity.

At step 204, the processor 110 continuously collects pressure data from the sensor array 140. The processor 110 also collects array positions associated with each sensor element 142, 144, 146. Accordingly, the pressure data may be associated with an array position corresponding to the respective sensor element 142, 144, 146.

At step 206, the processor 110 sends and stores the pressure data and the array positions on the cloud-based server 120. Alternatively, the processor 110 may store the pressure data and the array positions on the smart device 130. The processor 110 may also store a temporal component associated with the pressure data, indicative of a time at which the pressure data was recorded. Historic pressure data from previous activities may be stored in the cloud-based server 120 or the smart device 130.

At step 208, the processor 110 collects acceleration data from the accelerometer 160. The collection of the acceleration data in this embodiment occurs simultaneously to the collection of the pressure data at step 204. The acceleration data may be indicative of the golf club accelerating, for example during a golf shot. The processor 110 also collects clock data associated with the acceleration data, indicative of a time at which the acceleration data was collected.

At step 210, the processor 110 determines that the acceleration data exceeds a predetermined acceleration threshold. The predetermined acceleration threshold may be any suitable acceleration threshold selected by the user. Alternatively, an algorithm may be used to determine a predetermined acceleration threshold based on previous acceleration data collected from the user.

At step 212, in response to the determination outcome of step 210, the processor 110 identifies that an event of interest has taken place. The event of interest may be a golf shot.

At step 214, the processor 110 sends and stores to the cloud-based server 120, the acceleration data and the clock data associated with the acceleration data that has exceeded the predetermined acceleration threshold. Said acceleration data is associated with the event of interest. Historical acceleration data associated with previous events of interest occurring during previous activities and/or the same activity may also be stored in the cloud-based server 120. Alternatively, the acceleration data, the historical acceleration data and the clock may be stored on the smart device 130.

At step 216, the processor 110 discards, from the cloud-based server 120 and/or the smart device 130, the temporal component of the pressure data outside the clock data associated with the event of interest.

At step 218, the processor 110 determines a grip attribute associated with the pressure data corresponding to the event of interest. The grip attribute may be an average grip strength. Alternatively or additionally, the grip attribute may be a maximum grip strength. Alternatively or additionally, the grip attribute may be a temporal change in grip strength. Alternatively or additionally, the grip attribute may be a longitudinal change in grip strength. Alternatively or additionally, the grip attribute may be a lateral change in grip strength.

In the case of the grip attribute being an average grip strength, the processor 110 determines 220 an average pressure using the pressure data and clock data associated with the event of interest. Said average pressure may be used to determine an average force exerted by a user gripping the golf club during a golf shot. Said average force may be indicative of a user's average grip strength. A user's average grip strength during a first event of interest may be compared with the user's average grip strength during a second event of interest, in order to determine whether the user's average grip strength has weakened between events of interest. The comparison may be to a baseline grip strength. Said weakening may be indicative of an injury and/or a medical issue. Alternatively, strengthening of grip may occur due to therapeutic intervention or rehabilitation. The first and second event of interest may occur during the same session. Alternatively, the first and second event of interest may occur during different sessions.

In the case of the grip attribute being a maximum grip strength, the processor 110 determines 222 a maximum pressure using the pressure data associated with the event of interest. Said maximum pressure may be used to determine a maximum magnitude of force exerted by a user gripping the golf grip during a golf shot. Said magnitude of force may be indicative of a user's maximum grip strength. A user's maximum grip strength during a first event of interest may be compared with the user's maximum grip strength during a second event of interest in order to determine that the user's maximum grip strength decreased between events of interest. Said decrease in maximum grip strength may be indicative of an injury and/or a medical issue. If the user's maximum grip strength increased between events of interest, this may be indicative of the effects of therapeutic intervention or rehabilitation.

In the case of the grip attribute being a temporal change in grip strength, the processor 110 determines 224 a first time stamp of a first event of interest and a second time stamp of a second event of interest. The first time stamp may correspond to the second time stamp such that both time stamps correspond to a substantially similar segment of an activity. In particular, the first time stamp may correspond to a user initiating a golf swing during a first golf shot whilst the second time stamp may correspond to the user initiating a golf swing during a second shot. The processor may then compare pressure data corresponding to the first time stamp with pressure data corresponding to the second time stamp. Said comparison may be used to determine a change in grip strength between golf shots. Said change in grip strength may be indicative of an injury and/or a medical issue.

In the case of the grip attribute being a longitudinal change in grip strength, the processor 110 determines 226 a first longitudinal pressure and a second longitudinal pressure. The first longitudinal pressure may occur at a first array position and the second longitudinal pressure may occur at a second array position. That is, the first longitudinal pressure may be measured at a first location on the grip of the golf club and the second longitudinal pressure may be measured at a second location on the grip of the golf club. The first and second location may also be separated by a distance greater than a width of the user's fingers. Accordingly, the first location may correspond to a first finger and the second location may correspond to a second finger. The first and second location may share a common axis. The common axis may be a longitudinal axis of the grip of the golf club. Alternatively, the common axis may be substantially parallel to the longitudinal axis of the grip of the golf club. Accordingly, the first location may correspond to a portion of the user's first finger that is substantially similar to a portion of the user's second finger. A comparison between the first longitudinal pressure and the second longitudinal pressure may therefore indicate a difference in applied pressure by different fingers of the user and/or the user's palm. Said difference in applied pressure may indicate an injury and/or a medical issue.

In the case of the grip attribute being a lateral change in grip strength, the processor 110 determines 228 a first lateral pressure and a second lateral pressure. The first lateral pressure may occur at a first array position and the second lateral pressure may occur at a second array position. That is, the first lateral pressure may be measured at a first location on the grip of the golf club and the second lateral pressure may be measured at a second location on the grip of the golf club. The first and second location may share a common axis orthogonal to the longitudinal axis of the grip of the golf club. Accordingly, the first location may correspond to a first portion of the user's first finger that is substantially different to a second portion of the user's first finger. A comparison between the first lateral pressure and the second lateral pressure may therefore indicate a difference in applied pressure by a single finger of the user. Said difference in applied pressure may indicate a deviation in grip strength along the user's finger which in turn may indicate an injury and/or a medical issue. Said lateral change in grip strength may be determined for each finger.

Said grip attributes may be used alone or in combination in order to quantify the grip strength of a user.

At step 230, the processor may combine pressure data collected from additional grip analysis systems. For example, the processor may combine pressure data collected from an array of pressure sensors 140 arranged on a ski. Pressure data collected from different grip analysis systems may comprise different pressure patterns. Said pressure patterns may highlight grip strength weaknesses that may not be present during the use of a single grip analysis system. A change in grip pressure for a horizontal grip may indicate issues in some muscles that are not used in vertical grips. For example, in ski rackets or polls, a user applies force “vertically” (to a vertical instrument), while in trolleys used as walking aids the force is applied to the trolley handle “horizontally” (to a horizontal instrument). In the first case the applied pressure may be quite even on the grip circumference, while in the second the applied pressure may be focused on an upper portion of the grip. Accordingly, the use of multiple grip analysis systems may improve the precision of medical issue detection.

At step 232, the processor may apply a machine learning model to the pressure data collected from the grip analysis systems in order to predict a medical status of a user. The machine learning model may take into account additional variables such as user age, height, weight or any suitable variable for determining a medical status of a user. As pressure data from additional events of interest are collected, the predictive accuracy of the machine learning model may increase. Alternatively at step 232, the processor may apply other algorithms based on correlation and statistical metrics.

At step 234, the processor 110 may cause for display on the visual feedback device 150, a medical status.

The processor 110 shown in FIG. 1 may be adjacent to the array of pressure sensors 140, or remote from the array of pressure sensors 140. For example, the processor 110 and the array of pressure sensors 140 may both be positioned in the grip of a golf club. Alternatively, the array of pressure sensors 140 may be positioned in the grip of the golf club and the processor 110 may be positioned away from the golf club.

Although the server 120 is described as being cloud-based, it is to be understood that the server 120 may be located alternatively, such as centrally on a private network or locally on a local area network. Furthermore, although a smart phone and a smart watch have been given as examples of a smart device 130, it is to be understood that the smart device 130 may be any device capable of communicating with the processor 110.

The array of pressure sensors 140 may be arranged in a regular grid pattern. Alternatively, the array of pressure sensors 140 may be arranged in an irregular pattern. The array of pressure sensors 140 being configured to be arranged on an object to be gripped by a user may mean that the sensor elements 142, 144, 146 may be in, on or under a portion of the object. Furthermore, although the object has been described as a golf club, it is to be understood that any sporting equipment or other object may be used.

Although the pressure-applying elements are described as fingers, or palm portions, it is to be understood that the pressure-applying elements may be other items, human or non-human, such as portions of a robotic hand.

The method steps shown in flow diagram 200 of FIG. 2 are not limited to the steps shown and described above. Additional, or alternative, steps may be undertaken. For example, the machine learning algorithm can be trained first on a dataset (or datasets) incorporating grip strength data and health outcomes data.

1. A medical status analysis system comprising:
a sleeve positionable, in use, on an object configured to be gripped by a user;
a distributed array of pressure sensors arranged to detect a pressure applied to the sleeve;
an inertial sensor arranged to detect an inertial measurement of the object; and
a processor operable to:
detect, with the inertial sensor, an event of interest, by:
receiving inertial input data from the inertial sensor; and
determining that the event of interest has occurred based on a comparison between the inertial input data and a predetermined inertial threshold;
detect, with the array of pressure sensors, a grip of a user on the sleeve;
analyse the grip of the user on the sleeve, by:
receiving input data from the array of pressure sensors;
determining a grip attribute based on the input data; and
determining a medical status of the user based on the grip attribute; and
output the medical status corresponding to the user.

2. The medical status analysis system of claim 1, wherein the inertial sensor is one or more selected from the range of:
an accelerometer;
a gyroscope;
a tilt sensor; and
a magnetometer.

3. The medical status analysis system of claim 1, wherein the event of interest comprises:
inertial input data that has met the predetermined inertial threshold; and
a clock data;
wherein the clock data corresponds to the inertial input data.

4. The medical status analysis system of claim 1, wherein the input data comprises a temporal component.

5. The medical status analysis system of claim 4, wherein the processor is further operable to:
compare the temporal component of the input data to the clock data of the event of interest; and
output event data having a temporal component matching the event clock data

6. The medical status analysis system of claim 5, wherein the grip attribute is determined using the event data having the matching temporal component.

7. The medical status analysis system of claim 1, wherein the grip attribute is one or more selected from the range of:
an average grip strength;
a maximum grip strength;
a temporal change in grip strength;
a longitudinal change in grip strength across a length of the sleeve; and
a lateral change in grip strength across a width of the sleeve substantially perpendicular to a length of the sleeve.

8. The medical status analysis system of claim 7, wherein the processor is operable to determine the average grip strength by:
averaging the input data over the temporal component.

9. The medical status analysis system of claim 7, wherein the processor is operable to determine the maximum grip strength by:
calculating a maximum pressure comprised in the input data over the temporal component.

10. The medical status analysis system of claim 7, wherein the processor is operable to determine the temporal change in grip strength by:
determining a first pressure having a first time stamp corresponding to the temporal component;
determining a second pressure having a second time stamp corresponding to the temporal component; and
calculating a difference between the first pressure and the second pressure.

11. The medical status analysis system of claim 10, wherein the first time stamp corresponds to the second time stamp.

12. The medical status analysis system of claim 7, wherein the processor is operable to determine the longitudinal change in grip strength by:
determining a first longitudinal pressure at a first longitudinal position along a longitudinal axis of the sleeve; and
determining a second longitudinal pressure at a second longitudinal position spaced from the first longitudinal positon along the said longitudinal axis of the sleeve;
wherein determining the longitudinal change in grip strength comprises calculating a difference between the first longitudinal pressure and the second longitudinal pressure.

13. The medical status analysis system of claim 7, wherein the processor is operable to determine the lateral change in grip strength by:
determining a first lateral pressure at a first lateral position along an axis orthogonal to the longitudinal axis of the sleeve; and
determining a second lateral pressure at a second lateral position;
wherein determining the lateral change in grip strength comprises calculating a difference between the first lateral pressure and the second lateral pressure.

14. The medical status analysis system of claim 1, wherein the input data from the array of pressure sensors is stored on a remote server.

15. The medical status analysis system of claim 14, wherein the remote server is in communication with at least one other medical status analysis system.

16. The medical status analysis system of claim 1, wherein the processor is adjacent to the sleeve and is operatively connected to:
the array of pressure sensors; and
the inertial sensor.

17. A medical status analysis method comprising the steps:
detecting, by an array of pressure sensors, a grip of a user on a sleeve;
detecting, by an inertial sensor, an inertial measurement of the object;
detecting, with the inertial sensor, an event of interest, by:
receiving inertial input data from the inertial sensor; and
determining that the event of interest has occurred based on a comparison between the inertial input data and a predetermined inertial threshold;
analysing the grip of the user on the sleeve by:
receiving input data from the array of pressure sensors;
determining a grip attribute based on the input data;
determining a medical status of the user based on the grip attribute; and
outputting the medical status corresponding to the user.

18. A grip analysis system wherein the grip analysis system is the medical status analysis system of claim 1.
